(19) United States
(12) Patent Application Publication
(10) Pub. No.: US 2007/0280924 A1
(43) Pub. Date: Dec. 6, 2007

(54) Pharmaceutical Preparations For And Treatment Of Ocular Surface and Other Disorders

(76) Inventors: Julie Daniels, London (GB); Stephanie Watson, North Turramurra (AU); Gerd Geerling, Luebeck (DE); John Dart, London (GB)

Correspondence Address: Leon R. Yankwich;YANKWICH & ASSOCIATES, 201 Broadway, Cambridge, MA, 02139, US

(21) Appl. No.: 10/590,859

(22) PCT Filed: Mar. 2, 2005
(86) PCT No.: PCT/GB05/00806

§ 371 Date: Jul. 19, 2007

(30) Foreign Application Priority Data
Mar. 2, 2004 (GB) 0404693.4

(51) Int. Cl. A61K 38/40; A61K 31/56; A61K 38/47; A61K 47/16; A61K 47/26; A61P 27/02; A61K 47/22; A61K 47/10; A61K 38/03

(52) U.S. Cl. 424/94.61; 514/12; 514/13; 514/14; 514/169; 514/773; 514/777; 514/781; 514/788; 514/789

(57) ABSTRACT

A pharmaceutical preparation suitable for use in the eye, which comprises: (i) a pharmaceutically acceptable carrier suitable for use in the eye; (ii) one or more ingredients selected from factors and agents that promote any one or more of survival, health, cell attachment and normal differentiation of ocular surface epithelial cells and optionally factors and agents to prevent squamous metaplasia; (iii) one or more agents capable of altering the fluid properties of a tear film including at least one agent capable of establishing and/or maintaining a stable tear film and optionally one or more agents selected from opthalmological lubricating agents, viscosity enhancing agents and agents capable of reducing tear film evaporation; the factors and agents in component (ii) and (iii) being synthetic or recombinant or licensed for pharmaceutical use.

### INTRODUCTION

The present invention relates to pharmaceutical preparations and their use in the treatment and/or prophylaxis of dry eye conditions and other ocular surface disorders.

### BACKGROUND OF THE INVENTION

Integrity of the ocular surface is essential for visual acuity and ocular protection. Ocular surface disorders are a group of diseases and disorders of diverse pathogenesis, which result from the failure of the mechanisms responsible for maintaining a healthy ocular surface. Any condition or disorder in which the ocular surface is not a properly functioning unit is an ocular surface disorder.

The causes of ocular surface disorder may be nutritional, traumatic, iatrogenic, proliferative, may be secondary to lid abnormalities, may be caused by abnormal tear film, or may be neurotrophic. Trauma may be physical, chemical or thermal. Ocular surface disorders are often resistant to therapy. Some types of ocular surface disorders result from or cause dry or severely dry eyes, a condition also known as keratoconjunctivitis sicca. However, dry eye conditions may occur without causing or resulting from ocular surface disorders.

Two basic types of dry eye conditions are generally recognised, namely “tear deficient” dry eye, also known as “evaporative” dry eye, in which fewer tears are produced than by normal eyes, and “tear sufficient” dry eye, in which the volume of tears produced is apparently normal or approximately normal, but the constitution of the tears is such that they do not function properly.

The most common cause of tear sufficient dry eye is meibomian gland disease. The meibomian glands secrete lipids that affect the surface tension of the tears and hence their ability to wet the surface of the eye. In the absence of sufficient and/or suitable lipids the tears do not fulfil their function properly.

Tear deficient dry eye conditions include both Sjögren's dry eye and non-Sjögren's dry eye. When part of Sjögren's syndrome, dry eye condition is often severe. Non-Sjögren's dry eye is very common. Dry eye conditions, even when not associated with other pathologies, cause much discomfort and pain and predispose the eye to infection and may, in rare cases, cause corneal melting.

A wide variety of treatments have been proposed for ocular surface disorders and dry eye conditions. Such treatments include topical therapy, surgery and therapeutic contact lenses.

Current commercially available preparations for tear replacement therapy are ocular lubricants, which can improve tear volume and hydrodynamics. They are generally composed of electrolytes, water and agents that increase retention time on the ocular surface.

### SUMMARY OF THE INVENTION

The present invention provides a pharmaceutical preparation suitable for use in the eye, which comprises

(i) a pharmaceutically acceptable carrier suitable for use in the eye;

(ii) one or more ingredients selected from factors and agents that promote any one or more of survival, health, cell attachment and normal differentiation of ocular surface epithelial cells and optionally factors and agents that prevent squamous metaplasia;

(iii) one or more agents capable of altering the fluid properties of a tear film including at least one agent capable of establishing and/or maintaining a stable tear film and including one or more agents selected from opthalmological lubricating agents, viscosity enhancing agents and agents capable of reducing tear film evaporation;

the factors and agents in components (ii) and (iii) being synthetic or recombinant or licensed for pharmaceutical use.

This pharmaceutical preparation is called herein “an ocular surface medium” or “OS Medium”.

An ocular surface medium of the invention may also comprise (iv) one or more agents suitable for use in the treatment of an ocular surface disease, disorder or damage.

Such a preparation is called herein “a therapeutic ocular surface medium” or “TOS Medium”.

A pharmaceutical preparation of the invention comprising components (i), (ii) and (iii) may further comprise (v) one or more ingredients selected from factors and agents that promote any one or more of survival and maintenance of stem cell characteristics, growth of ocular surface stem cells, and survival, maintenance and differentiation of stem cell offspring in vitro or in vivo, the factors and agents being synthetic or recombinant or licensed for pharmaceutical use.

Such a preparation is called herein “a limbal stem cell medium” or “LSC Medium”.

A limbal stem cell medium of the invention, comprising components (i), (ii), (iii) and (v) may further comprise (iv) one or more agents suitable for use in the treatment of an ocular surface disease, disorder or damage.

Such a preparation is called herein “a therapeutic limbal stem cell therapeutic medium” or “TLSC Medium”.

Unless specified otherwise, the term “a pharmaceutical preparation of the present invention” is used herein generically to denote any preparation of the invention, that is to say, an ocular surface medium of the invention, a therapeutic ocular surface medium of the invention, a limbal stem cell medium of the invention and a therapeutic limbal stem cell medium of the invention.

The present invention also provides a pharmaceutical preparation of the invention for use as a medicament, for example, for use in treatment or prophylaxis of a dry eye condition or an ocular surface disorder. An ocular surface medium or therapeutic ocular surface medium of the invention may be used.

The present invention also provides a method for treatment or prophylaxis of a dry eye condition or an ocular surface disorder in a subject, which comprises administering a therapeutically effective amount of a pharmaceutical preparation of the invention to the affected eye of the subject. An ocular surface medium or therapeutic ocular surface medium of the invention may be used.

The present invention also provides a pharmaceutical preparation of the invention, in particular a limbal stem cell medium or a therapeutic limbal stem cell medium of the invention, for use in treatment or prophylaxis of a condition involving a deficiency or failure of limbal stem cells, or for post-operative therapy following surgery for limbal stem cell transplantation.

The present invention further provides a method for use in treatment or prophylaxis of a condition involving a deficiency or failure of limbal stem cells in an eye of a subject, or for post-operative therapy following surgery for limbal stem cell transplantation in an eye of a subject, which comprises administering a therapeutically effective amount of a pharmaceutical preparation of the invention, for example, a limbal stem cell medium or a therapeutic limbal stem cell medium of the invention, to the affected eye of the subject.

The present invention provides the use of an ocular surface medium or limbal stem cell medium of the present invention as a pharmaceutical vehicle or carrier for an opthalmological pharmaceutical composition.

The present invention also provides a opthalmological pharmaceutical composition that comprises a therapeutic agent and, as the or a pharmaceutical vehicle or carrier, an ocular surface medium or limbal stem cell medium of the present invention.

The present invention also provides a method of treating an ocular surface disorder in a subject in need of such a treatment comprising administering a therapeutically effective amount of a pharmaceutical preparation of the invention.

Preferably the ocular surface disorder is selected from scaring, ocular pemphigoid, persistent epithelial defect, acute ocular surface disorder, chronic ocular surface disease, infection or inflammation of the eye, neoplastic conditions of the eye and trauma to the eye.

Preferably said subject is a mammal. Preferably the mammal is a human. Alternatively the mammal may be a non-human animal. Non-human animals include animals raised for food, transport, hides, hair or fleece, for example, cattle, horses, sheep, goats, pigs; animals used for the production of a pharmaceutically useful agent, for example, recombinant proteins; stud and breeding animals; racehorses; and companion animals, for example, cats, dogs and small mammals.

Alternatively said subject may be a bird, for example, a bird raised for food or as a companion animal.

Definitions

The following terms used herein have meanings given below:

Cell attachment: Cell attachment means adhesion of cell to each other and to the basement membrane.

Dry eye condition and dry eye: The terms “dry eye condition” and “dry eye” are used herein to include all conditions characterised by deficient and/or defective tears. In such conditions the ocular surface is subjected to an aqueous deficiency. Dry eye may range from mild to severe, and may or may not be part of an ocular surface disorder or another disease condition. Some types of ocular surface disorders result from or cause dry or severely dry eyes, a condition also known as keratoconjunctivitis sicca. However, dry eye conditions may occur without causing or resulting from ocular surface disorders.

Growth: The term “growth” when used to describe a process that continues over a long period of time, generally implies an increase in total mass and volume accompanied by a proportional increase in number of cells. On a short term basis, the term “growth” can describe an increase in cell size (mass and volume) with no change in cell number.

Growth factor: Growth factor means a non-nutritive substance that does not participate in biosynthesis, metabolism or catalysis, but instead controls proliferation in a regulative manner.

Growth requirement: Growth requirement refers to anything that has a positive effect on cell multiplication.

Health: Health means the maintenance of the normal characteristics and function of the cell, and also maintenance of the cell phenotype.

Hormones: Hormones are chemical substances that are transmitted through body fluids and affect target cells at locations remote from the cells that produce them.

Multiplication and proliferation: Multiplication and proliferation both imply a net increase in cell number, with a corresponding increase in total mass and volume, such that both daughter cells become essentially identical to their parent cell.

Normal cells: Normal cells are cells that do not differ in any significant way from cells found in a healthy intact organism.

Normal differentiation: Normal differentiation means differentiation to the normal cell end point.

Nutrient: Nutrient refers to a chemical substance that is taken into a cell and utilised as a substrate in biosynthesis or energy metabolism, or else as a catalyst in one of those processes.

Ocular surface disorder: Any condition or disorder in which the ocular surface is not a properly functioning unit is an ocular surface disorder. Ocular surface disorders include diseases and disorders of diverse pathogenesis, which result from the failure of mechanisms responsible for maintaining a healthy ocular surface. The cause of an ocular surface disorder may be nutritional, iatrogenic, proliferative, may be secondary to lid abnormalities, or may be neurotrophic. Ocular surface disorders may result from damage to the ocular surface, for example, by surgery, by accidental trauma including physical, chemical and thermal trauma, by scarring, and also includes ocular pemphigoid. Some types of ocular surface disorders result from or cause dry or severely dry eyes.

Survival: “Survival” refers specifically to the maintenance of viability. In most case, survival implies retention of the ability to respond by multiplication when all growth requirements are satisfied.

Survival requirement: Survival requirement refers to any member of the set of minimal environmental conditions that must be provided in order for the cells in question to remain fully viable.

Synthetic and recombinant factors and agents: Synthetic and recombinant factors and agents are substances that have been produced by chemical synthesis or by recombinant DNA technology i.e. they have not been obtained from natural sources.

Tear break-up time (BUT): Tear break-up time (BUT) is the interval between a complete blink and the appearance of the first dry step on the coneal surface.

### DETAILED DESCRIPTION OF THE INVENTION

Pharmaceutical Preparations of the Invention

As stated above, the present invention provides a pharmaceutical preparation suitable for use in the eye, which comprises

(i) a pharmaceutically acceptable carrier suitable for use in the eye;

(ii) one or more ingredients selected from factors and agents that promote any one or more of survival, health, cell attachment and normal differentiation of ocular surface epithelial cells and, optionally, from factors and agents that prevent squamous metaplasia;

(iii) one or more agents capable of altering the fluid properties of a tear film including at least one agent capable of establishing and/or maintaining a stable tear film; and, optionally one or more agents selected from opthalmological lubricating agents, viscosity enhancing agents and agents capable of reducing tear film evaporation; the factors and agents in components (ii) and (iii) being synthetic or recombinant or licensed for pharmaceutical use.

This embodiment of the invention is called “an ocular surface medium” or “OS Medium”. Suitable carriers (i) and factors and agents for use in components (ii) and (iii) are described and exemplified in the section “Ingredients of pharmaceutical preparations of the invention” below.

An ocular surface medium of the invention may also comprise (iv) one or more agents suitable for use in treatment or prophylaxis of an ocular surface disorders or damage in addition to components (i) to (iii). Agents suitable for use in treatment or prophylaxis of ocular surface disorders include, for example, mydriatics, steroids, mucolytic agents, inhibitors of angiogenesis, antifibrotic agents, antimicrobial agents, and agents that reduce the accumulation of toxic by-products at the ocular surface. Further examples of agents suitable for use in component (v) are given in “Ingredients of pharmaceutical preparations of the invention” below.

A pharmaceutical preparation of the invention comprising components (i), (ii) and (iii) may further comprise (v) one or more ingredients selected from factors and agents that promote any one or more of survival and maintenance of stem cell characteristics, growth of ocular surface stem cells, and survival, maintenance and differentiation of stem cell offspring in vitro or in vivo.

Such a preparation is called herein “a limbal stem cell medium” or “LSC Medium”. Examples of ingredients suitable for use in component (v) are given in “Ingredients of pharmaceutical preparations of the invention” below.

A limbal stem cell medium of the invention, comprising components (i), (ii), (iii) and (v), may further comprise (iv) one or more agents suitable for use in treatment or prophylaxis of an ocular surface disorder.

Such a preparation is called herein “a therapeutic limbal stem cell therapeutic medium” or “TLSC Medium”.

Limbal stem cells, which occur in the limbus tissue at the junction of the cornea and the conjunctiva and/or in the formix, are the progenitors of the epithelial cells of the conjunctiva and cornea i.e. the ocular surface. Even partial failure of these stem cells to maintain healthy, normally differentiated ocular surface epithelial cells has severe consequences for the ocular surface.

Ingredients of Pharmaceutical Preparations of the Invention

Component (i): Component (i) of the pharmaceutical preparations of the invention is a pharmaceutically acceptable carrier suitable for use in the eye. Carriers suitable for use in the eye are well known and are described in pharmacopoeiae. They include suitably purified water for eye drops, and cream, gel and ointment bases for opthalmological compositions. For example, carbomers are often used as bases for gels, and paraffin and/or lanolin for ointments. The carrier may comprise one or more agents selected from tonicity agents for example, glucose; and buffering agents for example HEPES or bicarbonate.

Component (ii): All the pharmaceutical preparations of the present invention comprise one or more ingredients selected from factors and agents that promote any one or more of survival, maintenance, health, growth, migration, cell attachment and normal differentiation of epithelial cells and, optionally, that prevent squamous metaplasia.

Such agents may be selected from agents that provide metabolisable source of carbon, amino acids, growth factors, vitamins, antioxidants, mucin substitutes, bulk ions, trace elements, proteins and hormones, protease inhibitors, and anti-microbial agents.

Preferred ingredients in the various categories above are given below. Any selection of one or more ingredients from each category may be used, and any combination of ingredients may be used.

Preferably, a selection of ingredients includes at least one ingredient from each category.

Metabolisable Source of Carbon

Glucose, pyruvate, preferably glucose.

Amino Acids

Preferably all of the essential amino acids and, optionally, one or more of the non-essential amino acids, amino acids preferably being L-amino acids:

Essential amino acids: Arginine, Cysteine, Glutamine, Histidine, Isoleucine, Leucine, Lysine, Methionine, Phenylalanine, Threonine, Tryptophan, Valine

Non-essential amino acids: Alanine, Glycine, Asparagine, Aspartic acid, Glutamic acid, Proline, Serine, Tyrosine

Growth Factors

EGF (epithelial growth factor), HGF (hepatocyte growth factor), KGF (keratinocyte growth factor),

NGF (neuronal growth factor), glial-derived neurotophic factor), neurotrophins.

Antimicrobial Agents

Lactoferrin, lysozyme, defensins, secretory Immunoglobulin A

Hormones

Insulin

Vitamins

Vitamin C (ascorbic acid and salts thereof), and optionally any one or more of biotin, folic acid, lipoic acid, niacinamide, pantothenate, pyridoxine, riboflavin, thiamine, vitamin B12 and vitamin A.

Antioxidants

Tyrosine and/or glutathione.

Mucin Substitutes

Synthetic mucin substitutes and/or hyaluronic acid

Electrolytes

Comprising one or more electrolyte selected from bulk ions, for example, any one of more of sodium, potassium, calcium, chloride, bicarbonate, nitrate, sulphate, magnesium, and phosphate ions; and trace elements, for example, any one or more of copper, iron, manganese, molybdenum, nickel, selenium, silicon, tin, vanadium, and zinc

Organic Compounds

Any one or more of adenine, allantoin, choline, i-inositol, linoleate, putresceine, pyruvate, and thymidine.

Protease Inhibitors

Any one or more of tissue inhibitors of matrix metalloproteases (TIMPs), α1-antitrypsin, α2-macroglobulin, inter-α-antitrypsin, and α1-chymotrypsin.

Attachment Factors

Fibronectin

Agents that Reduce the Accumulation of Toxic Byproducts of Cell Metabolism

Chelators, free-radical scavengers.

In component (ii), a pharmaceutical preparation of the invention should generally comprise a metabolisable source of carbon, which is preferably glucose. Glucose is generally present at a concentration of from about 20 to about 1500 mg/l, preferably at about 26 mg/ml.

Lactoferrin is preferably present, for example, at a concentration of from about 0.2 to about 4 mg/ml, for example, about 1.5 mg/ml.

Lysozyme is a further preferred ingredient present at a concentration of about 0.2 to about 7.0 mg/ml more preferably at a concentration of about 0.4 to about 3.5 mg/ml, for example, about 1.5 mg/ml.

Vitamin C is preferably present, for example, at a concentration of from about 100 to about 500 μg/ml, for example, about 117 μg/ml for an ocular surface medium or a limbal surface medium and about 500 μg/ml for a therapeutic ocular surface medium.

Vitamin A is generally present. For an ocular surface medium or for a limbal surface medium the concentration is, for example, about from 10 to 20 ng/ml, for example, about 15 ng/ml. For a therapeutic ocular surface medium or a therapeutic limbal surface medium, the concentration is preferably higher, for example, a concentration of about 0.5 mg/ml would be suitable for use in a therapeutic medium for the treatment of alkali injury.

Epidermal growth factor is preferably present, for example, in a concentration of from about 0.1 to about 2 ng/ml, for example, about 1 ng/ml.

Tyrosine is preferably present, for example, in a concentration of from about 40 to about 100 μMolar, for example, at about 62 μMolar.

Glutathione is preferably present in addition to or as an alternative to tyrosine, for example, in a concentration of from about 50 to about 110 μMolar.

Sodium ions are preferably present, for example, in an amount of from about 140 to about 150 mEq/litre, for example, about 145 mEq/litre.

Potassium ions are preferably present, for example, in an amount of from about 20 to about 30 mEq/litre, for example, from about 24 to about 25 mEq/litre.

Calcium, chlorine, bicarbonate, nitrate, phosphate and sulphate ions are preferably present, calcium ions at a concentration of about 1.0 to about 2.0 mM, for example, about 1.5 mM, chlorine ions at a concentration of from about 120 to about 130 mM, for example, about 128 mM, bicarbonate ions at a concentration of from about 20 to about 30 mM, for example, about 26 mM, nitrate ions at a concentration of about 0.1 to about 0.2 mM, for example, from about 0.13 to about 0.14 mM, phosphate ions at a concentration of from about 0.15 to about 0.25 mM, for example, from about 0.20 to about 0.24 mM, and sulphate ions at a concentration of from about 0.35 to about 0.45 mM, for example, from about 0.38 to about 0.40 mM.

It may be advantageous for a preparation of the invention to comprises all the ingredients listed in this section, i.e. glucose, lactoferrin, lysozyme, EGF, tyrosine, glutathione, vitamin C, vitamin A and sodium, potassium, calcium, bicarbonate, nitrate, phosphoric and sulphate ions. The concentrations are preferably as set out above.

Component (iii): Component (iii) of a pharmaceutical preparation of the present invention comprises one or more agents capable of altering the fluid properties of a tear film including at least one agent capable of establishing and/or maintaining a stable tear film and optionally one or more agents selected from opthalmological lubricating agents, viscosity enhancing agents and agents capable of reducing tear film evaporation.

Agents capable of establishing and/or maintaining a stable tear film are known in the art and include various lipids, preferably polar lipids, for example sphingomyelin and phosphatidylcholibe, and lipoproteins, for example, ethanolamine and phosphoethanolamine. Alternatively or in addition meibomian gland secretions, or synthetic analogues thereof, or one or more components thereof may be used. Meibomian gland secretions include the protein lipocalin, which is an agent involved in establishing and/or maintaining a stable tear film.

Opthalmological lubricating agents, viscosity enhancing agents and agents capable of reducing tear film evaporation are also known in the art and include, for example, hypromellose (also known as hydroxypropylmethylcellulose), semisynthetic cellulose derivatives, methylcellulose, carbomers (for example, those sold under the brand name “Carbopol”), carmellose, polyvinyl alcohol, polyacrylic acid, povidone, dextran solutions, and viscoelastic agents, for example, hyaluronic acid, sodium hyaluronate and chondroitin sulphate.

According to certain embodiments of the invention, component (iii) preferably comprises hypromellose which has, according to one embodiment of the invention, been shown by the Inventors to have particularly good lubricant properties, as well as exhibiting desirable nutrient properties, low toxicity and showing good stability during storage. Preferably, such a pharmaceutical preparation comprises 0.2 to 0.4% hypromellose.

Component (iv): The optional component (iv) of a pharmaceutical preparation of the present invention comprises one or more agents suitable for use in treatment or prophylaxis of ocular surface disease, disorders or damage. Such agent include, for example, isoproterenol; corticosteroids, for example, hydrocortisone and dexamethasone; non-sterodial anti-inflamatory agents; mucolytic agents, for example, acetylcysteine; inhibitors of angiogenesis, for example, angiostatin, angiotensin and anti-VEGF; attachment factors, for example, fibronectin; antifibrotic agents, for example, anti-TGFβ; antimicrobial agents, for example, antibiotics, defensins, disinfectants, and antimicrobial agents found in normal tears, for example, lysozyme, lactoferrin and sigA; and agents that reduce the accumulation of toxic byproducts of cell metabolism, for example, chelators, free-radical scavengers and anti-oxidents, protease inhibitors, for example, matrix metalloprotease inhibitors, α1-antitrypsin, α2-macroglobulin, inter-α-trypsin, and α1-chymotrypsin; and vitamin A.

A pharmaceutical preparation of the present invention preferably does not contain a preservative, especially not benzalkonium chloride, which is toxic to ocular surface cells. The incorporation of anti-microbial agents, for example, any one or more of lactoferrin, lysozyme, defensins and sigA ensures the preparation can be used for the normal period of one month without microbial contamination, provided that the usual standards of hygiene are maintained. A non-preserved preparation is preferably stored at about 4° C. e.g. in a refrigerator.

Component (v): Component (v), which is present in the limbal stem cell preparations of the invention comprises one or more ingredients selected from factors and agents that promote any one or more of survival and maintenance of stem cell characteristics, growth of stem cells, and survival, maintenance and differentiation of stem cell offspring in vitro or in vivo. Such agents include EGF, basic FGF and NGF, as those factors stimulate the proliferation of limbal stem cells and their progenitors.

Optimal amounts, concentrations and ratios of the various ingredients of a pharmaceutical preparation of the present invention may be determined empirically, in accordance with known practice using in vitro and/or in vivo tests. Methods for isolating and culturing epithelial cells in vitro, for example, corneal and conjunctival epithelial cells, are well known, see for example, WO98/16629. Methods of determining ocular epithelial cell growth and differentiation in vivo using animal models are also known, for example, the well established Draize test. For ethical reasons, however, it is preferable to carry out as much validation as possible in vitro.

Detailed descriptions of ingredients suitable for use in pharmaceutical preparations of the present invention and an indication of appropriate concentrations and other factors are given in the Examples below. The person skilled in the art is able to modify any of the parameters in accordance with any particular requirement using routine procedures and common general knowledge, for example, as described above.

The pharmaceutical preparations of the present invention are suitable for administration to humans or to non-human animals, especially to humans. Non-human animals include animals raised for food, transport, hides, hair or fleece, for example, cattle, horses, sheep, goats, pigs and birds; animals used for the production of pharmaceutically useful agents, for example, recombinant proteins; stud and breeding animals; racehorses; and companion animals, for example, cats, dogs, small mammals and birds.

To comply with regulatory standards for preparations for human or veterinary use, the preparations should be free from ingredients obtained from humans or animals, in particular from blood, organs and glands, unless those ingredients are licensed for pharmaceutical use. The ingredients of a pharmaceutical preparation of the present invention should be synthetic or recombinant, or licensed for pharmaceutical use. Preferably the preparations should comply with the European Union transmissible spongiform encephalopathy (TSE) requirements for medicinal products as given in General Monograph 1483 and General Chapter 5.2.8 of the European Pharmacopia.

Formulations

A pharmaceutical preparation of the present invention is in a form suitable for use in the eye, for example, in the form of a solution, cream, ointment or gel.

Carriers suitable for use in the eye are well known and are described in pharmacopoeias. They include suitably purified water for drops, carbomer for gels, and paraffin and/or lanolin for ointments.

The pH of the pharmaceutical preparation is appropriate for its intended use. For example, eye drops may have a pH in the range of from 4.5 to 9.0, for example, from 6.0 to 9.0 preferably from 6.6 to 8.0, most preferably about 7.2. For treatment or prophylaxis of dry eye an alkaline pH, for example up to about 8.5, may be preferred. The osmolarity of eye drops is generally in the range from 100 to 350 mOsm, for example, from 120 to 320 mOsm, for example, from 150 to 350 mOsm, for example from 290 to 320 mOsm, for example, about 305 mOsm. The same pH and osmolarity ranges are generally used for creams, gels and ointments.

The surface tension of eye drops is preferably from about 40 dyne/cm to about 80 dyne/cm, for example, about 60 dyne/cm.

The contact angle (wetting angle) of the eye drops is preferably from about 20° to about 50°, for example, about 30°.

The viscosity of the eye drops is preferably from about 5 cps to about 50 cps, for example, about 10 cps.

Pharmaceutical preparations of the present invention should preferably produce clinically significant prolongation of tear break-up time (BUT). Tear BUT is dependant on tear film composition and anatomical factors, for example, lid-globe incongruity which may vary between individuals. As a general guide, a pharmaceutical preparations of the present invention should, when applied at a typical dose, for example, a drop of 50 μl, result in a prolongation of BUT of at least 20 minutes, preferably, at least 40 minutes, for example, about 50 minutes.

In the case of a solution, the ingredients of a pharmaceutical preparation of the invention may be dissolved in the carrier and filled into appropriate containers. Single dose units may be provided, for example, single dose plastic ampoules. Other formulations, for example, gels, creams and ointments are produced according to normal pharmaceutical practice. Such formulations may be provided in single dose units or in containers that enable single doses to be dispensed, for example, metered pumps. It may be advantageous to use a solution, especially when a therapeutic agent is present in the preparation, as it is generally easier to provide a unit dose with a solution than with a cream, gel or ointment. One drop of a solution is generally about 50 μL.

Therapeutic Utility of the Pharmaceutical Preparations of the Present Invention

As stated above, the pharmaceutical preparations of the present invention are useful in the treatment or prophylaxis and various eye conditions.

Ocular surface disorders are a group of disorders of diverse pathogenesis, in which disease results from the failure of the mechanisms responsible for maintaining a healthy ocular surface. The causes of the disorder may be nutritional, traumatic, iatrogenic, proliferative, or may be secondary to lid abnormalities, may be caused by abnormal tear film, or may be neurotrophic. The defects are often resistant to healing. Some types of ocular surface disorders result from or cause dry or severely dry eyes. Dry eye conditions that are not severe are generally called “dry eye” or “dry eye condition”. Severely dry eyes are a condition also known as keratoconjunctivitis sicca. However, dry eye conditions may occur without causing or resulting from ocular surface disorders. When part of Sjögren's syndrome, dry eye condition is often severe. Non-Sjögren's dry eye (“dry eye”) is very common. Dry eye conditions, even when not associated with other pathologies, cause much discomfort and pain and predispose the eye to infection. Any condition or disorder in which the ocular surface is not a properly functioning unit is an ocular surface disorder. Such disorders and conditions include dry eye, kerato-conjunctivitis sicca and Sjögren's syndrome, scarring, post-surgery conditions, and ocular pemphigoid, persistent epithelial defect, or acute or chronic ocular surface disease.

Limbal stem cells, which occur in the limbus tissue at the junction of the cornea and the conjunctiva and/or in the formix, are the progenitors of the epithelial cells of the conjunctiva and cornea, i.e. the ocular surface. Even partial failure of these stem cells to maintain healthy, normally differentiated ocular surface epithelial cells has severe consequences for the ocular surface.

An ocular surface medium of the invention, comprises

(i) a pharmaceutically acceptable carrier suitable for use in the eye;

(ii) one or more ingredients selected from factors and agents that promote any one or more of survival, health, cell attachment and normal differentiation of ocular surface epithelial cells and, optionally, from factors and agents that prevent squamous metaplasia;

(iii) one or more agents capable of altering the fluid properties of a tear film including at least one agent capable of establishing and/or maintaining a stable tear film; and, optionally one or more agents selected from opthalmological lubricating agents, viscosity enhancing agents and agents capable of reducing tear film evaporation; the factors and agents in components (ii) and (iii) being synthetic or recombinant or licensed for pharmaceutical use.

By promoting any one or more of survival, health, cell attachment and normal differentiation of ocular surface epithelial cells and optionally preventing squamous metaplasia while maintaining a stable tear film, an ocular surface medium of the invention actively promotes a normal, healthy ocular surface and may be used in treatment and/or prophylaxis of dry eye conditions and ocular surface disorders. Dry eye conditions include dry eye, kerato-conjunctivitis sicca and Sjögren's syndrome. Ocular surface disorders include scarring, post-surgery and other post-trauma conditions, ocular phemphigoid, persistent epithelial defect, and acute or chronic ocular surface disease.

A therapeutic ocular surface medium of the invention, which comprises (iv) one or more agents suitable for use in treatment or prophylaxis of ocular surface, disorders or damage in addition to components (i) to (iii) may be used in treatment or prophylaxis of ocular surface disorders and dry eye conditions in which there are or may be additional pathological conditions, for example, infection or inflammation, a neoplastic condition, trauma, or an autoimmune, degenerative, or iatrogenic condition. Further examples are post-surgery and other post-trauma conditions, for example, penetrating keratoplasty in eyes with a history of persistent epithelial defect (PED) and following large conjunctival autografts.

A limbal stem cell medium of the invention comprises

(v) one or more ingredients selected from factors and agents that promote any one or more of survival and maintenance of stem cell characteristics, growth of ocular surface stem cells, and survival, maintenance and differentiation of stem cell offspring in vitro or in vivo in addition to components (i) to (iii). A limbal stem cell medium of the invention may be used in treatment or prophylaxis of conditions that involve deficiencies or failure of limbal stem cells, for example, partial or complete limbal stem cell failure, and post-operative therapy following surgery for limbal stem cell transplantation.

A therapeutic limbal stem cell medium of the invention comprises component (iv) as defined above in addition to components (i) to (iii), and (v). A therapeutic limbal stem cell medium may be used in treatment or prophylaxis of the limbal stem cell conditions above in which there are or may be additional pathological conditions, for example, infection or inflammation, a neoplastic condition, trauma, or an autoimmune, degenerative, or iatrogenic condition. Further examples are post-surgery and other post-trauma conditions, for example, penetrating keratoplasty in eyes with a history of persistent epithelial defect (PED) and following large conjunctival autografts.

The present invention provides a pharmaceutical preparation of the invention for use as a medicament. The invention also provides the use of the various media defined above for the particular indications given as follows:

An ocular surface medium of the invention for use in treatment or prophylaxis of ocular surface disorders and dry eye conditions, including dry eye, kerato-conjunctivitis sicca and Sjögren's syndrome, scarring, post-surgery conditions, ocular pemphigoid, persistent epithelial defect, or acute or chronic ocular surface disease.

A therapeutic ocular surface medium of the invention for use in treatment or prophylaxis of ocular surface disorders and dry eye conditions, including dry eye, kerato-conjunctivitis sicca and Sjögren's syndrome, scarring, post-surgery conditions, ocular pemphigoid, persistent epithelial defect, or acute or chronic ocular surface disease in which there is or may be additional pathological conditions, for example, infection or inflammation, a neoplastic condition, trauma, or an autoimmune, degenerative, or iatrogenic condition. Further examples are post-surgery and other post-trauma conditions, for example, penetrating keratoplasty in eyes with a history of persistent epithelial defect and following large conjunctival autografts.

A limbal stem cell medium of the invention for use in treatment or prophylaxis of conditions that involve deficiencies or failure of limbal stem cells, for example, complete or partial limbal stem cell failure, and post-operative therapy following surgery for limbal stem cell transplantation.

A therapeutic limbal stem cell medium of the invention for use in treatment or prophylaxis of conditions that involve deficiencies or failure of limbal stem cells, for example, complete or partial limbal stem cell failure, and post-operative therapy following surgery for limbal stem cell transplantation, in which there is or may be additional pathological conditions, for example, infection or inflammation, a neoplastic condition, trauma, or an autoimmune, degenerative, or iatrogenic condition. Further examples are post-surgery and other post-trauma conditions, for example, penetrating keratoplasty in eyes with a history of PED large conjunctival autografts.

The invention also provides the use of a pharmaceutical preparation of the invention for the manufacture of a medicament for the various methods of treatment and prophylaxis set out above.

The present invention further provides methods of treatment or prophylaxis of the various conditions described above comprising applying the appropriate medium to the affected eye, see the Summary of the Invention.

The pharmaceutical preparations of the invention promote survival, maintenance, health, growth, migration, cell attachment and/or normal differentiation of ocular surface epithelial cells and prevent squamous metaplasia and are effective in treatment of ocular surface disorders and dry eye conditions. Previously proposed “artificial tears” are ocular lubricants, which may improve tear volume and hydrodynamics but which do not promote survival, maintenance, health, growth, migration, cell attachment and/or normal differentiation of ocular surface epithetical cells. Previously proposed treatments for ocular surface disorders and dry eye conditions, which include topical therapy, surgery and therapeutic contact lenses, have not proven satisfactory. Dry eye conditions, even if not severe, cause much discomfort and pain and predispose the eye to infection. Such conditions are common. The pharmaceutical preparations of the present invention, in particular the ocular surface medium and the therapeutic ocular surface medium, provide simple and effective treatment and prophylaxis for such conditions and for other ocular surface disorders.

The limbal stem cell media of the present invention promote survival and maintenance of stem cell characteristics, and/or growth of ocular surface stem cells, and/or survival, maintenance and differentiation of stem cell offspring in addition to promoting survival, maintenance, health, growth, migration, cell attachment and/or normal differentiation of ocular surface epithelial cells and preventing squamous metaplasia. The limbal stem cell media promote the regeneration of limbal cells and their differentiation into epithelial cells and also nurture and support the stem cells themselves and the cells into which they differentiate. The limbal stem cell media of the present invention provide simple and effective treatment and prophylaxis for conditions that involve deficiencies or failure of limbal stem cells, for example, partial or complete limbal stem cell failure, and post-operative therapy following surgery for limbal stem cell transplantation.

Use as a Vehicle for other Therapeutic Agents

A pharmaceutical preparation of the present invention may comprise a therapeutic agent, in particular a therapeutic agent that is useful for treating conditions that are or may be associated with dry eye or an ocular surface condition.

In addition, the present invention provides the use of an ocular surface medium or limbal stem cell medium of the present invention as a pharmaceutical vehicle or carrier for an opthalmological pharmaceutical composition.

The present invention also provides an opthalmological pharmaceutical composition that comprises a therapeutic agent and, as the or a pharmaceutical vehicle or carrier, an ocular surface medium or limbal stem cell medium of the present invention. Such therapeutic agents include, for example, anti-microbial agents such as antibiotics, antibacterials, antifungals, antivirals and disinfectants; anti-inflammatory agents such as corticosteroids and non-steroidal anti-inflamatories; anti-glaucoma agents to lower intraocular pressure such as sympathomimetics, beta-blockers, prostaglandin analogues, parasympathomimetic, and carbonic anhydrase inhibitors; mucolytics such as acetylcysteine; mydriatics and cycloplegics such as anti-muscarinics and sympathomimetics; and anti-allergy agents such as most cell stabilisers and antihistamines.

An advantage of using a pharmaceutical preparation of the present invention, in particular an ocular surface medium (OSM) or a limbal stem cell medium (LSCM) instead of a conventional carrier is that a pharmaceutical preparation of the present invention supports the one or more of survival, health, cell attachment and normal differentiation of ocular surface epithelial cells while maintaining a stable tear film, thereby actively promoting a normal, healthy ocular surface and, in the case of the limbal stem cell medium, supports the growth and differentiation of the ocular surface stem cells.

Many ophthalmic therapeutic agents have a deleterious effect on the ocular surface, for example, some are toxic or contain preservatives that are toxic to the corneal epithelium. However, if the condition that requires treatment is sufficiently severe, the risk of failure to treat the condition may outweigh possibility of damage to the ocular surface. By promoting the health and viability of the ocular surface, the use of an OSM or LSCM of the invention as a vehicle for such a therapeutic agent counteracts the adverse effects of the therapeutic agent and hence reduces the risk of damage.

Benzalkonium chloride, a preservative often used in opthalmological preparations, damages ocular surface cells. However, use of that preservative cannot always be avoided. By promoting the health and viability of the ocular surface, the use of an OCS or LSCM of the invention as a vehicle for in a composition comprising benzalkonium chloride counteracts the adverse effects of the preservative and hence reduces the risk of damage.

Use of a Pharmaceutical Preparation of the Invention for In Vitro Culture of Ocular Surface Epithelial Cells

A pharmaceutical preparation of the present invention may be used as an in vitro culture medium for ocular surface epithelial cells.

The following non-limiting Examples illustrate the invention.

### EXAMPLES

### Example 1

Ocular Surface Medium

An Ocular Surface Medium was formulated as eye drops as follows:

Preparation of Ocular Surface Medium Basic Mixture

Water suitable for use in eye drops (“water”) was measured out to 80% of the total desired volume. While gently stirring this water with a magnetic stirrer, the following were added: L-alanine (8.69 mg/L), L-arginine.HCl (406.70 mg/L), L-asparagine.HCl (12.74 mg/L), L-aspartic acid (3.86 mg/L), L-cysteine.HCl.H₂O (40.53 mg/L), L-glutamic acid (14.28 mg/L), L-glutamine (984.40 mg/L), glycine (7.34 mg/L), L-histidine.HCL.H₂O (48.64 mg/L), L-isoleucine (5.79 mg/L), L-leucine (126.60 mg/L), L-lysine.HCl (52.98 mg/L), L-methionine (13.03 mg/L), L-phenylalanine (9.65 mg/L), L-proline (33.39 mg/L), L-serine (121.80 mg/L), L-threonine (22.97 mg/L), L-tryptophan (8.98 mg/L), L-tyrosine-disodium salt (11.27 mg/L), L-valine (67.75 mg/L), biotin (0.019 mg/L), D-Ca⁺⁺-pantothenate (0.29 mg/L), choline chloride (13.51 mg/L), folic acid (0.772 mg/L), i-inositol (17.37 mg/L), niacinamide (0.039 mg/L), pyridoxine.HCL (0.058 mg/L), riboflavin (0.039 mg/L), thiamine.HCl (0.29 mg/L), vitamin B12 (0.396 mg/L), putrescine.2HCl (0.193 mg/L), D-glucose (1462.00 mg/L), KCl (108.10 mg/L), NaCl (6553.00 mg/L), thymidine (0.704 mg/L), adenine (23.16 mg/L), [4-(2-hydroxyethyl)-1-piperazineethanesulfonic acid], (HEPES, 3220.00 mg/L), lipoic acid (0.193 mg/L), sodium pyruvate (53.08 g), sodium acetate (290.50 mg/L), Na₂HPO₄ (274.10 mg/L), Na₂SO₄ (3.38 mg/L), and recombinant human insulin (5.00 mg/L).

A stock solution of ethanolamine.HCl was prepared in water at 976 mgL/L and 0.615 ml/L of this stock was added to the basic medium solution, to give a final concentration of ethanolamine.HCl of 0.60 mg/L.

A stock solution of phosphoethanolamine was prepared in water at 1408.00 mg/L and 0.1001 ml/L of this stock was added to the basic medium solution, to give a final concentration of phosphoethanolamine of 0.141 mg/L.

A stock solution of FeSO₄.7H₂O (41.70 mg/L), MgCl₂.6H₂O (18890 mg/L), CaCl₂.2H₂O and 0.207 CuSO₄.5H₂O (1343.5 mg/L) was prepared in water containing 0.5 mil/L concentrated HCl, and 9.660 ml of this stock solution was added to the basic medium solution, to give final concentrations of 0.403 mg/L FeSO₄.7H₂O, 182.50 mg/L MgCl₂.6H₂O, 12.98 mg/L CaCl₂.2H₂O and 0.002 mg/L CuSO₄.5H₂O.

A stock solution of ZnSO₄.7H₂O (137.68 mg/L) was prepared in water, and 0.9660 ml of this solution was added to the basic medium solution to give a final concentration of 0.133 mg/L ZnSO₄.7H₂O.

A stock solution containing Na₂SeO₃ (0.513 mg/L), (NH₄)₆Mo₇O₂₄.4H₂O (0.124 mg/L), NaSiO₃.9H₂O (14.2 mg/L), NiSO₄.6H₂O (0.013 mg/L), MnCl₂.4H₂O (0.002 mg/L), SnCl₂.2H₂O (0.011 mg/L) and NH₄VO₃ (0.059 mgfL) was prepared in water with 0.5 ml/L concentrated HCl, and 9.660 ml of this stock solution was added to the basic medium solution to give final concentrations of 0.00496 mg/L Na₂SeO₃, 0.00120 mg/L (NH₄)6Mo₇O₂₄.4H₂O, 0.137 mg/L NaSiO₃.9H₂O, 0.00013 mg/L NiSO₄.6H₂O, 0.00002 mg/L MnCl₂.4H₂O, 0.00011 mg/L SnCl₂.2H₂O and 0.00057 ml/L NH₄VO₃.

A stock of hydrocortisone was prepared at 370 mg/L in 95% ethanol, and 0.2 ml of this stock was added to the basic medium solution to give a final concentration of hydrocortisone of 0.074 mg/L.

A stock solution of sodium triiodothyronine (T3) was prepared at 67.00 mg/L in 70% ethanol, and 0.1 ml of this stock was added to the basic medium solution to give a final concentration of T3 of 0.0067 mg/L.

NaHCO₃ (1160 mg/L) was added to the basic medium solution, and the pH of the solution was then adjusted with HCl to 7.2±0.25 and the volume adjusted to the full desired volume with water. The osmolality was determined to be 290±15 mOsm.

The mixture was then sterile filtered through a low protein binding filter, under appropriate conditions, bottled and stored under diminished light conditions at 4° C. until use.

### Preparation of the Supplement

To a sterile solution of Dulbecco's Phosphate Buffered Saline (DPBS) or water suitable for eye drops the following were added while gently stirring: ascorbic acid phosphate, magnesium salt (50 mg/L), acidic FGF (2.5 mg/L), sodium salt of recombinant human heparin (5000 USP units/L) and recombinant human EGF (0.1 mg/L).

A stock solution of isoproterenol.HCl (1,000,000 mg/L) was prepared in DPBS or water containing 50 mg/L ascorbic acid, and 1.25 ml/L of this solution was added to the above, to form a 500× formulation of the supplement.

This 500× solution was then sterile filtered through a low protein binding filter, and added to the basic medium mixture or aliquotted and stored at −20 to −80° C. until use.

Preparation of the Eye Props

The supplement was added to the mixture of the basic medium at a ratio of 1:500 by volume for use as eye drops. The resulting eye drops were stored at 4° C. under diminished light conditions until use.

The drops may be used every two hours (eight times per day).

### Example 2

A pharmaceutical preparation having the formulation given in Example 1 was tested by three healthy volunteers. Drops of the solution were administered to the eye every 2 hours (eight times per day) hours for up to a week. No adverse reactions occurred. The drops were described as “comfortable”.

### Example 3

Ocular Surface Medium

The data below gives a list of components for an ocular surface medium for treatment of dry eye which medium is an alternative to that given in Example 1. Preferred concentrations of each component and an approximate indication of a preferred range of concentrations of each component are given. Such a medium was made up in a similar way to that exemplified in Example 1.

MEDIUM

| | Concentration | |
|---|---|---|
| Component | (mg/litre) | Range (mg/litre) |
| Proteins | | |
| Lysozyme | 3000 | 200-6900 |
| Lactoferrin | 2000 | 400-3400 |
| sIgA | 1000 | 20-4500 |
| Tri-iodothyronine Sodium | 0.0067 | ±0.0003 |
| Zinc Insulin Human | 5 | ±0.3 |
| Amino Acids | | |
| Essential amino acids | | |
| L-Arginine•Hydrochloride | 406.7 | ±20.3 |
| L-Cysteine•Hydrochloride•H₂O | 40.53 | ±2.03 |
| L-Glutamine | 984.4 | ±49.2 |
| L-Histidine Hydrochloride•H₂O | 48.64 | ±2.43 |
| L-Isoleucine | 5.79 | ±0.29 |
| L-Leucine | 126.6 | ±6.3 |
| L-Lysine Hydrochloride | 52.98 | ±2.65 |
| L-Methionine | 13.03 | ±0.65 |
| L-Phenylalanine | 9.65 | ±0.48 |
| L-Threonine | 22.97 | ±1.15 |
| L-Tryptophan | 8.98 | ±0.45 |
| L-Valine | 67.75 | ±3.39 |
| Non-essential amino acids | | |
| L-Alanine | 8.69 | ±0.43 |
| Glycine | 7.34 | ±0.37 |
| L-Asparagine | 12.74 | ±0.64 |
| L-Aspartic Acid | 3.86 | ±0.19 |
| L-Glutamic Acid | 14.28 | ±0.71 |
| L-Proline | 33.39 | ±1.67 |
| L-Serine | 121.8 | ±6.1 |
| Taurine | 143.75 | ±7.20 |
| L-Tyrosine Disodium | 11.27 | ±0.56 |
| Vitamins | | |
| Ascorbic Acid-2-Phosphate | 50 | ±3 |
| Biotin | 0.019 | ±0.001 |
| Folic Acid | 0.772 | ±0.039 |
| Niacinamide | 0.039 | ±0.002 |
| D-Calcium Pantothenate | 0.29 | ±0.02 |
| Pyridoxine•Hydrochloride | 0.058 | ±0.003 |
| Riboflavin | 0.039 | ±0.002 |
| Thiamine•Hydrochloride | 0.29 | ±0.0015 |
| Vitamine B₁₂ | 0.396 | ±0.020 |
| Antioxidents | | |
| L-tyrosine disodium | see amino-acids | |
| Taurine | See amino-acids | |
| Glutathione | 32.88 | ±1.64 |
| Lipoic Acid | 0.193 | ±0.097 |
| Carbohydrate | | |
| D-Glucose | 1462 | ±146 |
| Lipids | | |
| Phosphorylethanolamine | 0.141 | ±0.007 |
| Ethanolamine | 0.6 | ±0.03 |
| Electrolytes | | |
| Bulk Inorganic ions | | |
| Sodium Chloride | 6553 | ±328 |
| Potassium Chloride | 108.1 | ±5.4 |
| Sodium Sulphate | 3.38 | ±0.17 |
| Calcium Chloride•2H₂O | 3.54 | ±0.18 |
| Sodium Bicarbonate | 1160 | ±116 |
| Magnesium Chloride•6H₂O | 182.5 | ±9.1 |
| Disodium Phosphate Dibasic | 274.1 | ±13.7 |
| Trace elements | | |
| Cupric Sulphate•5H₂0 | 0.002 | ±0.0001 |
| Ferrous Sulphate•7H₂O⁺ | 0.403 | ±0.02 |
| Manganese Chloride•4H₂O | 0.00002 | ±0.000001 |
| Ammonium Molybdate•4H₂O | 0.001 | ±0.00005 |
| Nickel Sulphate•6H₂O | 0.00013 | ±0.00001 |
| Sodium Selenite | 0.005 | ±0.00025 |
| Sodium Metasilicate•9H₂O | 0.137 | ±0.007 |
| Stannous Chloride•2H₂O | 0.00011 | ±0.00001 |
| Ammonium Metavanadate | 0.00057 | ±0.00003 |
| Zinc Sulphate•7H₂O | 0.133 | ±0.003 |
| Other organic components | | |
| Sodium Acetate | 290.5 | ±14.5 |
| Adenine | 23.16 | ±1.16 |
| Choline Chloride | 13.51 | ±0.68 |
| i-Inositol | 17.37 | ±0.87 |
| Linoleate | | |
| Putrescine•Dihydrochloride | 0.193 | ±0.010 |
| Sodium Pyruvate | 53.08 | ±2.65 |
| Thymidine | 0.704 | ±0.035 |
| Protease inhibitors | | |
| Tissue inhibitors of matrix | 50 | ±50 |
| Metalloproteinases (TIMPs) | | |
| Other therapeutic agents | | |
| Hydrocortisone | 0.074 | ±0.004 |
| Excipients | | |
| Buffers | | |
| HEPES Ultra Pure | 3220 | ±322 |
| Bicarbonate | As needed | See physical |
| | to adjust pH | properties |
| Tonicity agents | | |
| Glucose | As needed to adjust | See physical |
| | tonicity | properties |
| Sodium chloride | As needed to adjust | See physical |
| | tonicity | properties |

SUPPLEMENT FOR 1:500 DILUTION IN MEDIUM

| | Concentration | |
|---|---|---|
| Component | (mg/litre) | Range (mg/litre) |
| Vitamin | | |
| Ascorbic Acid 2-Phosphate | 500 | ±25 |
| Growth Factors | | |
| Recombinant EGF Human | 0.2 | ±0.02 |
| Recombinant HGF Human | 5 | ±0.25 |
| Recombinant KGF Human | 5 | ±0.25 |
| Acidic FGF Human | 2.5 | ±0.1 |
| Other therapeutic agents | | |
| Isoproterenol Hydrochloride | 125 | ±6 |
| Electrolytes | | |
| Bulk Inorganic ions | | |
| Sodium Chloride | 8000 | ±400 |
| Potassium Chloride | 200 | ±10 |
| Sodium Phosphate Dibasic | 2160 | ±108 |
| Potassium Phosphate Monobasic | 200 | ±10 |

The above medium when made in accordance with this example and with the supplement added at 1:500 dilution had the measured physical properties listed below. Also listed are ranges of acceptable values.

| Property | Measured values | Acceptable Range |
|---|---|---|
| Osmolality (Osmotic pressure) | 305 mOsm/kg | 290-320 mOsm/kg |
| | (=0.95% sodium | |
| | chloride) | |
| pH | 7.2 | 6.6-8 |
| Surface tension | 60 dyne/cm | 40-80 dyne/cm |
| Prolongation of BUT | 50 | 40-90 minutes |
| Angle of contact | 30° | 20-50° |
| Viscosity | 10 cps | 5-50 cps |
| Dose (single eye drop) | 50 microlitres | ±15 microlitres |

In vitro testing of the above medium also showed that it was able to support the viability of a human epithelial cell line and primary cultures of human corneal epithelial cells in vitro. Outgrowth of human corneal epithelial cells from cultured limbal explants was also supported by this medium. The wound healing activities of human corneal fibroblasts were found to be moderated by this medium compared to their activity in human serum. Such moderation may be a desirable characteristic in circumstances where it is desirable to avoid excess cell proliferation or where it is desirable to limit the formation of scar tissue.

### Example 4

The data given below gives a list of components for a further alternative ocular surface medium for treatment of dry eye. Such a medium was made up in a similar way to that exemplified in Example 1.

MEDIUM

| | Preferred | |
|---|---|---|
| | Concentration | Acceptable Range |
| Component | (mg/litre) | (mg/litre) |
| Proteins | | |
| Tri-iodothyronine Sodium | 0.0067 | ±0.0003 |
| Zinc Insulin Human | 5 | ±0.3 |
| Amino Acids | | |
| Essential amino acids | | |
| L-Arginine•Hydrochloride | 406.7 | ±20.3 |
| L-Cysteine•Hydrochloride•H₂O | 40.53 | ±2.03 |
| L-Glutamine | 984.4 | ±49.2 |
| L-Histidine•Hydrochloride•H₂O | 48.64 | ±2.43 |
| L-Isoleucine | 5.79 | ±0.29 |
| L-Leucine | 126.6 | ±6.3 |
| L-Lysine•Hydrochloride | 52.98 | ±2.65 |
| L-Methionine | 13.03 | ±0.65 |
| L-Phenylalanine | 9.65 | ±0.48 |
| L-Threonine | 22.97 | ±1.15 |
| L-Tryptophan | 8.98 | ±0.45 |
| L-Valine | 67.75 | ±3.39 |
| Non-essential amino acids | | |
| L-Alanine | 8.69 | ±0.43 |
| Glycine | 7.34 | ±0.37 |
| L-Asparagine | 12.74 | ±0.64 |
| L-Aspartic Acid | 3.86 | ±0.19 |
| L-Glutamic Acid | 14.28 | ±0.71 |
| L-Proline | 33.39 | ±1.67 |
| L-Serine | 121.8 | ±6.1 |
| L-Tyrosine Disodium | 11.27 | ±0.56 |
| | Concentration | |
| Component | (mg/litre) | Range (mg/litre) |
| Vitamins | | |
| Ascorbic Acid-2-Phosphate | 50 | ±3 |
| Biotin | 0.019 | ±0.001 |
| Folic Acid | 0.772 | ±0.039 |
| Niacinamide | 0.039 | ±0.002 |
| D-Calcium Pantothenate | 0.29 | ±0.02 |
| Pyridoxine•Hydrochloride | 0.058 | ±0.003 |
| Riboflavin | 0.039 | ±0.002 |
| Thiamine•Hydrochloride | 0.29 | ±0.0015 |
| Vitamine B₁₂ | 0.396 | ±0.020 |
| Antioxidants | | |
| L-Tyrosine Disodium | see amino-acids | |
| Lipoic Acid | 0.193 | ±0.097 |
| Carbohydrate | | |
| D-Glucose | 1462 | ±146 |
| Lipids | | |
| Phosphorylethanolamine | 0.141 | ±0.007 |
| Ethanolamine | 0.6 | ±0.03 |
| Electrolytes | | |
| Bulk inorganic ions | | |
| Sodium Chloride | 6553 | ±328 |
| Potassium Chloride | 108.1 | ±5.4 |
| Sodium Sulphate | 3.38 | ±0.17 |
| Calcium Chloride•2H₂O | 3.54 | ±0.18 |
| Sodium Bicarbonate | 1160 | ±116 |
| Ferrous Sulphate•7H₂O | 0.403 | ±0.02 |
| Magnesium Chloride•6H₂O | 182.5 | ±9.1 |
| Disodium Phosphate Dibasic | 274.1 | ±13.7 |
| Trace elements | | |
| Cupric Sulphate 5H₂0 | 0.002 | ±0.0001 |
| Ferrous Sulphate•7H₂O | 0.403 | ±0.02 |
| Manganese Chloride•4H₂O | 0.00002 | ±0.000001 |
| Ammonium Molybdate•4H₂O | 0.001 | ±0.00005 |
| Nickel Sulphate•6H₂O | 0.00013 | ±0.00001 |
| Sodium Selenite | 0.005 | ±0.00025 |
| Sodium Metasilicate•9H₂O | 0.137 | ±0.007 |
| Stannous Chloride•2H₂O | 0.00011 | ±0.000006 |
| Ammonium Metavanadate | 0.00057 | ±0.00003 |
| Zinc Sulphate•7H₂O | 0.133 | ±0.003 |
| Other organic components | | |
| Sodium Acetate | 290.5 | ±14.5 |
| Adenine | 23.16 | ±1.16 |
| Choline Chloride | 13.51 | ±0.68 |
| i-Inositol | 17.37 | ±0.87 |
| Putrescine•Dihydrochloride | 0.193 | ±0.010 |
| Sodium Pyruvate | 53.08 | ±2.65 |
| Thymidine | 0.704 | ±0.035 |
| Other therapeutic agents | | |
| Hydrocortisone | 0.074 | ±0.004 |
| Excipients | | |
| Buffers | | |
| HEPES Ultra Pure | 3220 | ±322 |
| Bicarbonate | see electrolytes | |
| pH Indicator | | |
| Phenol Red | 1.158 | ±0.058 |
| Tonicity agents | | |
| Glucose | see carbohydrate | |
| Sodium chloride | see electrolytes | |

SUPPLEMENT FOR 1:500 DILUTION IN MEDIUM

| | Concentration | |
|---|---|---|
| Component | (mg/litre) | Range (mg/litre) |
| Vitamins | | |
| Ascorbic Acid 2-Phosphate | 50 | ±2.5 |
| Growth factors | | |
| Recombinant EGF Human | 0.10 | ±0.01 |
| Recombinant Acidic FGF | 2.5 | ±0.1 |
| Other therapeutic agents | | |
| Isoproterenol Hydrochloride | 125 | ±6 |
| Electrolytes | | |
| Bulk inorganic ions | | |
| Sodium Chloride | 8000 | ±400 |
| Potassium Chloride | 200 | ±10 |
| Sodium Phosphate Dibasic | 2160 | ±108 |
| Potassium Phosphate Monobasic | 200 | ±10 |

### Example 5

Ocular Surface Repair Medium

The data below gives a list of components for a therapeutic ocular surface repair medium particularly suitable for the treatment of the following:
Persistent epithelial defect
Acute or chronic ocular surface disease
Post-operative treatment of penetrating keratoplasty in eyes with history of PED and post-operative treatment of large conjunctival autografts

The composition of both the medium and the supplement is as given in Example 3 with the following variations and additions:

MEDIUM

| Component | Concentration (mg/l) | Range (mg/litre) |
|---|---|---|
| Vitamins | | |
| Vitamin C (ascorbic acid) | 100000 | ±5000 |
| Proteinase inhibitors | | |
| Tissue inhibitors of matrix | 50 | ±50 |
| Metalloproteinases (TIMPs) | | |

SUPPLEMENT FOR 1:500 DILUTION IN MEDIUM

| Growth Factors | | |
|---|---|---|
| Recombinant EGF Human | 0.5 | ±0.05 |
| Recombinant HGF Human | 5 | ±0.25 |
| Recombinant KGF Human | 5 | ±0.25 |
| Recombinant Acidic FGF | 2.5 | ±0.1 |
| Recombinant Anti-TGFbeta e.g. CAT-152 | 10 | ±0.5 |
| antibody Human | | |
| Neurotrophins | | |
| NGF | 100 | 20-200 |
| GDNF | 100 | ±5 |
| Attachment factors | | |
| Fibronectin | 500 | 100-3500 |
| Other therapeutic agents | | |
| Isoproterenol Hydrochloride | 125 | ±6 |
| Electrolytes | | |
| Bulk inorganic ions | | |
| Sodium Chloride | 8000 | ±400 |
| Potassium Chloride | 200 | ±10 |
| Sodium Phosphate Dibasic | 2160 | ±108 |
| Potassium Phosphate Monobasic | 200 | ±10 |

### Example 6

Limbal Stem Cell Medium

The data below gives a list of components for a medium for treatment of limbal stem cell failure or dysfunction and post-operative limbal stem cell transplant

The composition of both the medium and the supplement is as given in Example 3 with the following additions and variations:

| | | |
|---|---|---|
| | Concentration | Range |
| Component | (mg/litre) | (mg/litre) |

MEDIUM

| | | |
|---|---|---|
| Vitamins | | |
| Vitamin A (retinoic acid) | 500 | ±25 |
| Proteinase inhibitors | | |
| Tissue inhibitors of matrix | 50 | ±50 |
| Metalloproteases (TIMPs) | | |

SUPPLEMENT

| | | |
|---|---|---|
| Growth Factors | | |
| Recombinant EGF Human | 0.2 | ±0.02 |
| Recombinant HGF Human | 5 | ±0.05 |
| Recombinant KGF Human | 5 | ±0.05 |
| Recombinant Basic FGF Human | 2.5 | ±0.1 |
| Recombinant Anti-TGF beta | 10 | ±1 |
| e.g. CAT-152 human antibody | | |
| Other therapeutic agents | | |
| Isoproterenol Hydrochloride | 125 | ±6 |
| Electrolytes | | |
| Bulk inorganic ions | | |
| Sodium Chloride | 8000 | ±400 |
| Potassium Chloride | 200 | ±10 |
| Sodium Phosphate Dibasic | 2160 | ±108 |
| Potassium Phosphate Monobasic | 200 | ±10 |

### Example 7

Dry Eye Clinical Trial

The clinical trial described in this Example demonstrates the efficacy of an ocular surface medium made in accordance with the present invention in the treatment of dry eye.

Patient Data

Study Population

patients were recruited from Corneal and External disease clinics at Moorfields Eye Hospital with moderate to severe dry eye. 8 patients were female and 2 patients were male. Both eyes of each patient were used in the trial.

| | |
|---|---|
| Age: | 52.90 ± 18.45 years |
| | range 18-77 |
| — | median 53 |

No patients were lost to follow-up and none withdrew from the study.

The disease profiles of the patients were as follows:

Sjögrens Syndrome

Primary (1) and secondary (5)
   Non-Sjögrens Aqueous Tear Deficiency (5)

Systemic Diseases
Rheumatoid arthritis (5)
Ocular Cicatricial Pemphigoid (OCP) (1)
Steven-Johnson syndrome (3)
Atopy (1)
Other autoimmune disease (2)
   Systemic Therapy
Corticosteroids, oral (1)
Immunosuppression, oral (2)
   Corneal Graft in Study Eye
Penetrating (2)
   Methods
   Clinical Scoring

Clinical condition of the study eyes were assessed by the following techniques:

Rose Bengal staining: Rose Bengal was instilled into the eyes. The eyes were divided up into 6 standardized regions and each region was given a score from 0 to 3 on the basis of the condition of that region with a score of 0 being the best and a score of 3 being the worst. The scores for the regions of each eye were then added together to give a total score for each eye out of a possible total score of 18.

Fluorescein staining: Fluorescein was instilled into the eyes. The eyes were divided up into 5 standardised regions and each region was given a score from 0 to 3 on the basis of the condition of that region with a score of 0 being the best and a score of 3 being the worst. The scores for the regions of each eye were then added together to give a total score for each eye out of a possible total score of 15.

Schirmer's test: Schirmer's test was carried out for 5 minutes without anaesthetic. The length of a strip of wet filter paper by tears during this time was recorded in mm.

Tear break-up time (BUT): The time for a dry spot to appear on the ocular surface following a full blink was measured with a stop watch and recorded in seconds.

Symptom score: Patients were asked to score the severity of symptoms on the following scale:

| | |
|---|---|
| Grade 0: | No symptoms. |
| Grade 1: | Symptoms were mild and they did not make me |
| | uncomfortable. |
| Grade 2: | Symptoms were moderate and they did make me |
| | uncomfortable, but did not interfere with my activities. |
| Grade 3: | Symptoms were severe and they did make me uncomfortable, |
| | but did not interfere with my activities. |
| Grade 4: | Symptoms were very severe, they did make uncomfortable |
| | and they did interfere with my activities. |

The following symptoms were scored:

Dryness

Foreign body sensation (sensation of sand or gravel in the eye)

Blurred vision

Discomfort

Photophobia

The scores for each symptom were added together to give a total symptom score out of a possible total score of 20.

Facial analogue score Mace score): The patient was shown a series of 10 faces ranging from “happy” (scoring 1 point) to “sad” (scoring 10 points) and asked “which face best describes your condition?”.

Conjunctival injection: The level of conjunctival injection (“blood shot eyes”) was graded from 0 to 4, grade 4 being the most severe, in accordance with the Corneal and Contact Lens Research Unity (CCLRU) grades.

Blepharitis: A score out of a total possible score of 12 was calculated for each eye based on the number of symptoms noted in each eye. Each symptom scored 1 point. The symptoms observed were:

| | | |
|---|---|---|
| Anterior lid margin: | Grease | Skin scales |
| | | Collarettes |
| | | Frank ulcers |
| | | Loss of lashes |
| | | Inflammation |
| Posterior lid margin: | Cheesey secretions | Blocked glands >⅓ |
| | | Telangieclasia |
| | | Meibomitis |
| | | Notched lid margin |
| | | Active chalazia |

Intra Ocular Pressure (IOP): IOP was measured and recorded as mmtlg.

Best Corrected Visual Acuity (BCVA): BCVA was measured and scored as follows:

1=6/6

2=6/9

3=6/12

4=6/18

5=6/24

6=6/36

7=6/60

8=3/60

9=1/60

10=HM,CF

11=PL

12=NPL

Baseline findings

| | | | | |
|---|---|---|---|---|
| RB staining | 12.9 ± 4.77 | (mean ± SD) | range 5-18 | median 13.5 |
| Fluorescein | 11.2 ± 3.88 | (mean ± SD) | range 6-15 | median 12.5 |
| Schirmer's | 1.20 ± 1.398 | (mean ± SD) | range 0-4 | median 1 |
| BUT | 1.20 ± 0.919 | (mean ± SD) | range 0-3 | median 1 |
| Symptom score | 15.60 ± 3.502 | (mean ± SD) | range 9-20 | median 15 |
| Face score | 7.33 2.062 | (mean ± SD) | range 3-9 | median 8 |
| Conjunctival | 3.0 ± 0.816 | (mean ± SD) | range 2-4 | median 3 |
| injection | | | | |
| Blepharitis | 1.90 ± 1.449 | (mean ± SD) | range 0-4 | median 2 |
| IOP | 14.0 ± 5.696 | (mean ± SD) | range 7-26 | median 13 |
| BCVA | 5.20 ± 3.458 | (mean ± SD) | range 1-10 | median 5 |

RB = Rose Bengal,
BUT = prolongation of tear break-up time,
IOP = Intra Ocular Pressure,
BCVA = best corrected visual acuity,
Schirmer's = Schirmer's test.

Intervention

The ocular surface medium of Example 1 administered to both eyes eight times per day for one month. Patients were assessed for subjective symptoms and objective signs on enrolment, week one, week 2 and on completion of trial at week 4.

Analysis

Changes in subjective and objective variables from baseline were analysed. The data were analysed on an “intent to treat” basis. For efficacy variables only the data from the worse eye was analysed. The worse eye was defined as that with the lowest score in Schirmer's test and the worse sum of corneal and conjunctival staining. If both eyes are similar the right was used.

All safety analyses included both eyes.

The Wilcoxon rank-sum test was used to assess differences between the 2 groups with respect to change from baseline in all primary and secondary efficacy variables at each follow-up visit.

Quantitative data was analysed by paired sample t-tests for the parametric data. Independent non-parametric data was analysed via a Mann-Whitney U test to compare 2 groups.

Results

Primary Outcome

Improvement in rose Bengal (RB) staining by 3 or more points from the baseline to completion of trial at week 4 in study eye (worse eye at baseline) was found in 7 of 10 patients in the study eye.

Secondary Outcomes

Subjective symptoms of dry eye were improved in all 10 patients. Significant improvement from enrolment to week 4 was seen for dryness for foreign body sensation, discomfort, photophobia and face score. No significant change from enrolment to week 4 was seen for blepharitis score, tear film break-up time prolongation, fluorescein staining score, Schirmer's test and intra ocular pressure (IOP).

Wilcoxon Signed Rank Test for Paired Samples

Symptoms in Study Eye, Enrolment Visit Compared to Baseline

| Symptom in | Improved | No change | Worse | |
|---|---|---|---|---|
| study eye | (n) | (n) | (n) | p |
| Dryness | 7 | 3 | 0 | 0.0104 |
| FB sensation | 8 | 0 | 2 | 0.0073 |
| Blurred vision | 1 | 3 | 6 | 0.3809 |
| Discomfort | 7 | 0 | 3 | 0.0106 |
| Photophobia | 7 | 1 | 2 | 0.0289 |
| Global symptom | 10 | 0 | 0 | 0.0049 |
| Face score | 8 | 0 | 1 | 0.0089 |

Signs in Study Eye, Enrolment Visit Compared to Baseline

| Sign in study eye | Improved (n) | No change (n) | Worse (n) | p |
|---|---|---|---|---|
| BCVA | 2 | 3 | 5 | 0.7404 |
| Fluorescein | 4 | 1 | 5 | 0.1232 |
| Rose Bengal | 9 | 1 | 0 | 0.0122 |
| Schirmers | 3 | 4 | 3 | 0.599 |
| BUT | 2 | 6 | 2 | 1.0 |
| IOP | 4 | 2 | 4 | 0.889 |
| Cojunctival | 7 | 3 | 0 | 0.008 |
| injection | | | | |
| Blepharitis | 5 | 1 | 4 | 0.952 |

Successful Cases (RB Score Improved by 3 or More Points from Enrolment to Week 4)
Patients with rheumatoid arthritis (5) and also with Sjögren's syndrome secondary (3)
Steven-Johnson syndrome (2)
Schirmer's test (length of filter paper strip wet by tears given in mm) 0 mm (2), 1 mm (2), 2 mm (1), 3 mm (1), 4 mm (1)
   Failure Cases (RB Score Showed Improvement of Less than 3 Points from Enrolment to Week 4)
Patients with OCP (1). Female age 54 on systemic immunosuppression. Schirmer's 0 m. RB score in study eye was 16 and change in R^{B} score was 2.
Steven-Johnson's syndrome and atopy (1). Female 18 years old. Schirmer's 0. R^{B} score in study eye was 9 and change in RB score was −3.
Primary Sjögren's syndrome (1). Female age 39 years. Schirmer's was 1 mm. RB score in study eye was 14 and change in RB score was 2.
   Safety Data (Analysed in both Eyes with Wilcoxon Signed Ranks test)

BCVA: No significant change in best-corrected visual acuity from baseline to week 4 in study eye (p=0.891) or in fellow eye (p=0.705).

IOP: No significant change in IOP from baseline to week 4 in study eye (p=0.889) or in fellow eye (p=0.482).

Conjunctival injection: Significantly reduced from baseline to week 4 in study eye (p=0.008) and in fellow eye (p=0.014).

Cataract: No patients developed cataract during the study period in the study or fellow eye. One patient underwent cataract extraction and intraocular lens implantation in the fellow eye during the trial without alteration to the topical or systemic ocular therapy.

Adverse events: One patient complained of blurred vision and discomfort whilst reading at week 2 and 3, the trail was continued and the symptoms resolved.

### CONCLUSION

A subjective improvement was seen in all 10 patients and 7 of these patients had signs of an objective improvement. There were no serious adverse events and no significant change in visual acuity, intraocular pressure nor lens opacity. The application of an ocular surface medium in accordance with the present invention was thus found to be an efficacious and safe therapy for ocular surface disorders.

### Example 8

Comparison of Various Viscosity Enhancing and Nutrient Agents

Various viscosity enhancing agents, in a range of concentrations, were used to supplement a physiologically compatible ocular surface medium according to the invention. The properties of the resultant media were then investigated in vitro.

Materials and Methods

Hypromellose, Carbopol or sodium hyaluronate were used to supplement an ocular surface medium at concentrations ranging from 0.0001% to 0.4%. The pH and osmolarity of all of the resultant formulations were controlled and adjusted to be within physiological range. The biophysical properties, i.e. viscosity and surface tension, were determined with a rheometer and an electronic manometer. Two human corneal epithelial cell lines (HCE-T and CEPI-17-CI.4) and two human conjunctival epithelial cells were used to investigate cell proliferation, viability and migration in response to the formulations by means of a luminescence based ATP-assay, a calcein AM/EthD-1 assay and a colony dispersion assay. All solutions were stored for three months at 4° C. and then retested to assess stability.

Results

The viscosity of the non-supplemented medium was 0.75 mPa.sec at shear rates of 1.7 to 128.5 s⁻¹. Hypromellose, Carbopol and sodium hyaluronate increased the viscosity of the medium significantly at all the concentrations, but only 0.2% and 0.4% hypromellose and 0.4% sodium hyaluronate supplemented medium showed both moderate viscosity and non-Newtonian behaviour. The viscosity of hypromellose-supplemented medium remained stable for three months when stored at 4° C. In contrast, the viscosity media supplemented with carbopol or sodium hyaluronate changed significantly. The surface tension of media supplemented with hypromellose showed a surface tension similar to tears of about 50 mN/m. For comparison the surface tension of water is about 70 mN/m. Media supplemented with 0.2% or 0.4% hypromellose also showed superior ability at supporting cell growth and cell migration.

1. A pharmaceutical preparation suitable for use in the eye, which comprises:
(i) a pharmaceutically-acceptable carrier suitable for use in the eye;
(ii) one or more ingredients selected from factors and agents that promote any one or more of survival, health, cell attachment and normal differentiation of ocular surface epithelial cells and optionally factors and agents that prevent squamous metaplasia;
(iii) one or more agents capable of altering the fluid properties of a tear film including at least one agent capable of establishing or maintaining a stable tear film and optionally one or more agents selected from the group consisting of opthalmological lubricating agents, viscosity enhancing agents and agents capable of reducing tear film evaporation;
and wherein the factors and agents in components (ii) and (iii) are synthetic or recombinant or licensed for pharmaceutical use.

2. The pharmaceutical preparation as claimed in claim 1, further comprising:
(iv) one or more agents suitable for use in the treatment or prophylaxis of an ocular surface disease, disorder, or damage.

3. The pharmaceutical preparation as claimed in claim 1, further comprising:
(v) one or more ingredients selected from the group consisting of factors and agents that promote survival and maintenance of stem cell characteristics, growth of ocular surface stem cells, and survival, maintenance and differentiation of stem cell offspring in vitro or in vivo, and wherein the factors and agents are synthetic or recombinant or licensed for pharmaceutical use.

4. The pharmaceutical preparation as claimed in claim 2, wherein the one or more of the agents (iv) suitable for use in the treatment or prophylaxis of an ocular surface disease, disorder or damage is selected from the group consisting of:
mydriatics agents, steroids, mucolytic agents, inhibitors of angiogenesis, attachment factors, antifibrotic agents, antimicrobial agents, anti-glaucoma agents, and agents that reduce the accumulation of toxic by-products of cell metabolism.

5. The pharmaceutical preparation as claimed in claim 1, wherein component (i) comprises one or more agents selected from the group consisting of:
purified water for eye drops, cream bases for opthalmological compositions, gel bases for opthalmological compositions and ointment bases for opthalmological compositions.

6. The pharmaceutical preparation as claimed in claim 1, wherein component (ii) comprises one or more agents selected from the group consisting of:
agents that provide a metabolisable source of carbon, amino acids, growth factors, vitamins, antioxidants, mucin substitutes, bulk ions, trace elements, proteins, hormones, protease inhibitors, and anti-microbial agents.

7. The pharmaceutical preparation as claimed in claim 1, wherein the agent capable of establishing or maintaining a stable tear film is selected from the group consisting of:
lipids, lipoproteins, and meibomian gland secretions, and synthetic analogues thereof.

8. The pharmaceutical preparation as claimed in claim 1, wherein the opthalmological lubricating agent, viscosity enhancing agent, or an agent capable of reducing tear film evaporation is selected from the group consisting of:
hypromellose, Semisynthetic cellulose derivatives, methylcellulose, hydroxyprophylmethylcellulose, carbomer, carmellose, polyvinyl alcohol, polyacrylic acid, povidone, dextran solutions, hyaluronic acid and chondroitin sulphate.

9. The pharmaceutical preparation as claimed in claim 1, wherein said preparation does not contain benzalkonium chloride.

10. The pharmaceutical preparation as claimed in claim 4, wherein the anti-microbial agent is selected from the group consisting of:
lactoferrin, lysozyme, defensin and sIgA;
and wherein the preparation may be kept at 4 degrees Celsius for up to one month without microbial contamination.

11. The pharmaceutical preparation according to claim 1, wherein the preparation has a pH of from 6.6 to 8.0.

12. The pharmaceutical preparation as claimed in claim 1, wherein the preparation is in the form of a solution for use as eye drops.

13. The pharmaceutical preparation as claimed in claim 1, wherein the preparation is in the form of a cream, ointment or gel.

14. The pharmaceutical preparation as claimed in claim 12, wherein said solution for use as eye drops has an osmolarity of from 290 mOsm to 320 mOsm.

15. The pharmaceutical preparation as claimed in claim 12, wherein said solution for use as eye drops has a surface tension in the range of from 40 dyne/cm to 80 dyne/cm.

16. The pharmaceutical preparation as claimed in claim 12, wherein said solution for use as eye drops has a viscosity of from 5 cps to 50 cps.

17. The pharmaceutical preparation as claimed in claim 1, wherein said preparation is in a single dose container.

18. (canceled)

19. (canceled)

20. The pharmaceutical preparation as claimed in claim 1, wherein said ocular surface disorder is selected from the group consisting of:
dry eye, severely dry eye, scarring, ocular pemphigoid, persistent epithelial defect, acute ocular surface disease, chronic ocular surface disease, infection or inflammation of the eye, neoplastic conditions of the eye and trauma to the eye.

21. (canceled)

22. A method of treating an ocular surface disorder in a subject in need of such treatment comprising administering to said subject a therapeutically effective amount of a pharmaceutical preparation as claimed in claim 1.

23. The method as claimed in claim 22, wherein the ocular surface disorder is selected from the group consisting of dry eye, severely dry eye, scarring, ocular pemphigoid, persistent epithelial defect, acute ocular surface disease, chronic ocular surface disease, infection or inflammation of the eye, neoplastic conditions of the eye and trauma to the eye.

24. The method as claimed in claim 22, wherein the subject is a mammal.

25. The method as claimed in claim 24, wherein the mammal is a human.

26. (canceled)

27. (canceled)
